(19) United States
(12) Patent Application Publication
(10) Pub. No.: US 2011/0192215 A1
(43) Pub. Date: Aug. 11, 2011

(54) Analytical System for In-Line Analysis of Post-Combustion Capture Solvents

(75) Inventor: Alan Finlay, West Byfleet (GB)

(73) Assignee: Microsaic Systems Limited, Surrey (GB)

(21) Appl. No.: 13/020,994

(22) Filed: Feb. 4, 2011

(30) Foreign Application Priority Data
Feb. 5, 2010 (GB) GB1001901.6

(51) Int. Cl. G01N 30/72

(52) U.S. Cl. 73/23.37

(57) ABSTRACT

A device and method is described for direct analysis of solvents used to chemically bind with CO₂ present in flue gases, and for the monitoring of large-scale CO₂ solvent-capture reaction to improve process efficiency, thereby reducing the cost of CO₂ capture.

### CROSS-REFERENCE TO RELATED APPLICATIONS

This application claims the benefit of Great Britain Patent Application No. 1001901.6 filed on Feb. 5, 2010.

### TECHNICAL FIELD OF THE INVENTION

The present invention relates to CO₂ capture systems and in particular to a method and system for direct analysis of solvents used to chemically bind with CO₂ present in flue gases, and for the monitoring of large-scale CO₂ solvent-capture reaction to improve process efficiency, thereby reducing the cost of CO₂ capture.

### BACKGROUND OF THE INVENTION

In recent years, interest in the development of efficient processes for the capture of CO₂ from coal-flue gas or other carbon fuel sources has increasingly been driven by the concerns about the impact of rising CO₂ emissions from fixed sources. Solvent Scrubbing, also known as “sweetening” or acid gas removal, was originally developed to remove H₂S and CO₂ from gases in natural gas processing plants and other industries. Solvents based on amines are commonly used in CO₂ capture (CC) plants. The amine solvent reacts with flue gases to strip out greenhouse gases such as methane and CO₂ by chemically binding with them to form carbamates and other reaction products. The chemically-bound CO₂ may then be outgassed under conditions of elevated temperature and pressure, and may be collected, transported and stored.

In a solvent-based CC plant, the processes taking place are complex and the chemical reaction mechanisms involved are not well understood. Factors affecting the efficiency of CO₂ capture plants include solvent breakdown rates, the mechanisms that chemically bind the CO₂, the formation of intermediate reaction products, process transients and the non-measurement of toxic by-products. These factors could be critical to improving the energy balance of a CO₂ capture plant and their environmental impact, and therefore pivotal to reducing CC plant operational costs to commercially feasible levels.

Various amines-based solvents have been proposed for CO₂ capture processes. While some research has been conducted on amine-based CO₂ capture in the past, little has been done to characterise its chemical composition in real-time. Likewise, the chemical processes leading to the degradation of solvents, plant corrosion and the formation of toxic products are not well understood and have not been monitored on capture plants. Amines undergo a variety of degradation processes and form various salts, and the solvent is gradually consumed over time. Capture efficiency falls, and running costs are introduced due to energy imbalances and the requirement for solvent replenishment. Amine degradation is a major concern for long-term full-scale CC plant operation not only because of economics but increasingly because of environmental concerns.

The formation of heat stable salts leads to excessive foaming, reducing gas liquid contact and thus reducing the amount, and increasing the specific energy, of CO₂ captured on a single pass through the absorber, as well as leading to increased solvent loss rates and the formation of potentially corrosive species.

So far, solvents have only been analysed off-line using conventional laboratory-based mass spectrometer instruments. While this off-line detection of reaction products such as carbamates demonstrates the feasibility of monitoring reaction composition, the opportunity to intervene and alter reaction conditions (e.g. temperature, pressure, pH, flow rate, solvent composition) in order to maintain capture efficiency during load changes is lost. Clearly, failure to capture CO₂ during load changes is unacceptable if limits of 90% capture become set in legislation, especially when permits will have to be purchased for the lost CO₂. Moreover, considering that solvent-based CO₂ capture plant is expected to add 20% to 35% to energy prices the economic value of further efficiency losses will be considerable.

As mentioned above to date, solvent analysis has been performed off-line in analytical laboratories, often using techniques such as gas chromatography (GC) or gas chromatography mass spectrometry (GC-MS). These analytical laboratories are often located off-site. Samples are collected infrequently from the rich and lean solvent streams, often months apart. The time lag between collecting the sample, analysing it and reporting results can be hours to days depending on the location of the analytical instrumentation. Consequently, the opportunity to intervene and to adjust process parameters to optimise CC plant efficiency is lost. As the quality of the solvent degrades, its capacity to absorb CO₂ deteriorates and the energy required by the PCC process rises, increasing operating costs. Therefore monitoring the solvent quality through in-line analysis of its chemical composition will permit the adjustment of conditions to maintain solvent quality, preserving the energy balance and optimising operating costs.

Accordingly there is a need for improved monitoring of CO₂ solvent-capture process composition.

### SUMMARY OF THE INVENTION

To overcome these and other problems, a system and methodology is described for providing a direct analysis of the CO₂ capture reaction occurring within a CO₂ capture plant. In accordance with a preferred arrangement a mass spectrometer is coupled to a solvent-based CO₂ capture reaction chamber. The mass spectrometer (MS) of the invention is used to directly monitor the chemical composition of the solvent during the solvent scrubbing or CO₂ capture process. The chemical composition, in particular the formation of chemically bound CO₂ as carbamates, may be used to calculate the percentage of CO₂ captured and the overall yield of the process. In accordance with the present teaching, data on chemical composition may be used in closed-loop control of the solvent-capture process. Information of this kind could be used to optimise reaction conditions for capture efficiency. Similarly, real-time compositional data could be used as feedback to a closed-loop control system to adjust parameters such as temperature, flow, pH, solvent dilution, solvent replenishment, flow rates and pressure etc. for optimal process performance. This capability would be particularly important because of changes in the composition of flue-gases due to combustion of coal mixes of varying quality. Monitoring the composition of the solvent-based mixtures used in a CO₂ capture processes would also permit measurement of the rate of solvent consumption and its degradation mechanisms.

In accordance with the present teaching a MS coupled to a solvent-based capture plant could be used to optimise absorber column conditions and accelerate reactions. A MS system is described that when coupled to a solvent-based post-combustion CO₂ capture (PCC) plant, monitors changes in the composition of solvents such as monoethanolamine (MEA), AEPD (2-amino-2-ethyl-1,3-propanediol), AMP (2-amino-2-methyl-1-propanol), AMPD (2-amino-2-methyl-1,3-propanediol), DEA (diethanolamine), MDEA (methyldiethanolamine), PZ (piperazine) and THAM (tris-(hydroxymethyl)aminomethane) in real-time. In accordance with the present teaching it is possible to track degradation of the solvents in a PCC plant. By measuring the reaction conditions that affect solvent consumption ratessolvent consumption, solvent replenishment, energy and operating costs can be minimised.

In a first embodiment, the sytem comprises a MS consisting of an inlet for extracting a sample from a fluid stream, an ion source, a mass analyser and an ion counter. The inlet of the MS of the invention is fluidically coupled to a solvent-based, CO₂ scrubbing plant and is used to monitor the chemical composition of the CO₂ capture process. The ion source functions by transforming neutral molecules of the species of interest into charged particles called ions. This ion has a mass to charge ratio that corresponds to its molecular mass. To avoid fragmentation or distruction of volatile molecules, and to permit the easy identification of the species of interest based on their molecular ions, the MS system preferably incorporates a ‘soft’ ionisation source and a mass analyser. A soft ionisation source limits fragmentation of the molecules of interest. The soft ionisation source may be based on, but not limited to, electrospray ionisation (ESI), nanospray ionisation, chemical ionisation, secondary eletrospray ionisation (SESI), atmospheric pressure chemical ionisation (APCI), DART, DESI, MALDI, atmospheric pressure photoionisation (APPI) or glow discharge ionisation. The analyser of the MS system may be an ion trap, time of flight, quadrupole, magnetic sector, orbital ion trap, linear ion trap, rectilinear ion trap, cross-field, cycloidal or rotational field mass analyser. The MS system of the invention is used for in-line analysis of CO₂ capture reactions and may be based on liquid chromatography mass spectrometry (LC-MS) or GC-MS. The MS system of the invention is coupled to a CO₂ capture reactor and used to monitor reactor composition to provide degradation kinetics for solvents such as MEA and related amines. The chemical species of interest are extracted in fluid samples. This MS system generates chemical composition data in real-time that can be linked to process parameters such as temperature, amine concentration, CO₂ loading, pH and the influence of reactor vessel materials.

In another embodiment, the MS system of the invention is a compact MS that is configured to be coupled fluidically to a CO₂ capture plant. By fluidically coupling the MS to the CO₂ capture plant sample may be extracted from a fluid stream in the PCC process. The sample may be taken from rich or lean solvent streams, or from a suitable sample port on the absorption column provided within such CO₂ capture plants. The sample will be appreciated as being a fluid mixture containing particulate and may require filtration. Before injection into the MS systems, a solution may be made-up from a reservoir of suitable solvent using a make-up pump. In a first arrangement, the system of the present teaching utilizes a soft ionisation source to couple the sample solution to a MS. The soft ionisation source ionises the chemical species as they elute and the MS identifies the species based on the mass to charge ratios and mass spectra of the ions. The MS analyses the chemical composition of the reactor fluid and detects carbamate species formed by the reaction of solvent and CO₂ for online measurement of CO₂ loading.

In another a chromatographic separator is used to couple a soft ionisation source to the sample solution. The chromatographic module separates the chemical constituent of the mixture of the sample solution so that they elute individually into a soft ionisation source. The chromatograhic module may be based on GC, LC or supercritical fluid chromatography (SFC). The soft ionisation source ionises the chemical species as they elute and the MS identifies the species based on the mass to charge ratios and mass spectra of the ions. The MS detects the chemical composition of the sample solution and detects carbamate species formed by the reaction of solvent and CO₂ for online measurement of CO₂ loading.

In another embodiment of an in-line analytical system a sample is extracted from the rich or lean solvent streams of the PCC process, or from a point along the absorption column. A sample solution is made-up and injected onto a chromatographic column by means of a sample injector and a sample loop. The sample loop measures out a known volume of sample solution, and injects it onto the column by means of a valve and injection pump. The chromatographic module be based on GC, LC or SFC. The chemical constituents of the mixture of the sample solution are separated and elute individually into a soft ionisation source where their molecules are transformed into ions. The soft ionisation source preserves chemical bonds and limits molecular fragmentation, minimising chemical interference, easing interpretation of mass spectra and thereby improving system selectivity. The ions are analysed by the MS and mass spectra are used to ‘name’ the chemical species of the sample. The presence of molecular ions in the spectra may be used to identify chemical compounds of interest by means of their molecular mass.

In another embodiment, the analytical system of the invention is a compact MS system that is coupled fluidically with a PCC plant and forms part of its control system. A fluid sample is extracted, using a fluid interface to the PCC plant, from the rich or lean solvent streams of the PCC process, or from a point along the absorption column. A sample solution is made-up as necessary and the fluid sample is ionised by means of a soft ionisation source. The soft ionisation source preserves chemical bonds and limits molecular fragmentation, minimising chemical interference, easing interpretation of mass spectra and thereby improving system selectivity. The use of soft ionisation may avoid the need for chromatography in the case of less complex mixtures composed of known substances. The ions are transported into a vacuum system by means of a vacuum interface and analysed by the mass analyser. Ion current from the mass analyser is collected and measured by an ion counter. The signal from the ion counter is acquired and processed by a computer and used to display mass spectra on an analytical display. The mass analyser may also be operated in selected ion monitoring (SIM) mode where a handful of ions are of interest, each representing a certain species of interest. Mass spectra are used to ‘name’ the chemical species of the sample. The presence of molecular ions in the spectra may be used to identify chemical compounds of interest by means of their molecular mass. The computer may linked to the control system of the PCC and used to transmit data on chemical composition to the control system. The data link may be on-line, forming part of a closed feedback loop, or off-line so that data is monitored by process technicians in a control room. The system of the invention monitors starter materials, intermediate products and reaction products and may provide feedback to a closed-loop control system. The MS system analyzes the chemical composition of the solvent in real-time, thus generating data for the concentration of each chemical present in the mixture. The MS tool continuously measures the relative concentration of starter materials (e.g. MEA, H₂O, CO₂) and reaction products (e.g. carbamates). Data provided by MS monitoring tool is used to measure the efficiency and yield of the capture reaction at any given moment. The MS system data is used to adjust process conditions in order to accelerate reactions, to minimise solvent degradation and reduce waste products.

In another embodiment, ions generated by the soft ionisation source may be separated by their drift time along the drift tube of an ion mobility spectrometer (IMS). The IMS effects some separation of the ions by means of permitting them to drift in a strong, a potentially varying, electric field. The IMS may be a field-asymmetric ion mobility spectrometer (FAIMS). A vacuum interface couples the IMS to the a mass analyser inside a vacuum chamber. The ions are transported into a vacuum system by means of a vacuum interface and analysed by the mass analyser. Ion current from the mass analyser is collected and measured by an ion counter. The signal from the ion counter is acquired and processed by a computer and used to display mass spectra on an analytical display. The mass analyser may also be operated in selected ion monitoring (SIM) mode where a handful of ions are of interest, each representing a certain species of interest. Mass spectra are used to ‘name’ the chemical species of the sample. The presence of molecular ions in the spectra may be used to identify chemical compounds of interest by means of their molecular mass. The computer may linked to the control system of the PCC and used to transmit data on chemical composition to the control system. The data link may be on-line, forming part of a closed feedback loop, or off-line so that data is monitored by process technicians in a control room. The system of the invention monitors starter materials, intermediate products and reaction products and may provide feedback to a closed-loop control system. The MS system analyzes the chemical composition of the solvent in real-time, thus generating data for the concentration of each chemical present in the mixture. The MS tool continuously measures the relative concentration of starter materials and reaction products and data provided by MS in-line monitoring system is used to measure the efficiency and yield of the capture reaction at any given moment through adjusting process conditions in order to accelerate reactions, to minimise solvent degradation and reduce waste products.

In another embodiment, the analytical system of the invention is a compact MS system that is coupled fluidically with a PCC plant and forms part of its control system. A fluid sample is extracted from the rich or lean solvent streams of the PCC process, or from a point along the absorption column. A sample solution is made-up as necessary and the fluid sample is separated by gas chromatography (GC). The eluent is ionised by means of a atmospheric pressure ionisation source. The atmospheric pressure ionisation source may be a suitable soft ionisation source such as ESI, SESI, APCI or APPI that preserves chemical bonds and limits molecular fragmentation, minimising chemical interference, easing interpretation of mass spectra and thereby improving system selectivity. The vacuum interface is an atmospheric pressure interface (API) that couples the atmospheric pressure ionisation source to the a mass analyser inside a vacuum chamber. The ions are transported into a vacuum system by means of a vacuum interface and analysed by the mass analyser. Ion current from the mass analyser is collected and measured by an ion counter. The signal from the ion counter is acquired and processed by a computer and used to display mass spectra on an analytical display. The mass analyser may also be operated in selected ion monitoring (SIM) mode where a handful of certain ions are of interest, each representing a chemical species of interest. Mass spectra are used to ‘name’ the chemical species of the sample. The presence of molecular ions in the spectra may be used to identify chemical compounds of interest by means of their molecular mass. The computer may linked to the control system of the PCC and used to transmit data on chemical composition to the control system. The data link may be on-line, forming part of a closed feedback loop, or off-line so that data is monitored by process technicians in a control room. The system of the invention monitors starter materials, intermediate products and reaction products and may provide feedback to a closed-loop control system. The MS system analyzes the chemical composition of the solvent in real-time, thus generating data for the concentration of each chemical present in the mixture. The MS tool continuously measures the relative concentration of starter materials and reaction products and data provided by MS in-line monitoring system is used to measure the efficiency and yield of the capture reaction at any given moment through adjusting process conditions in order to accelerate reactions, to minimise solvent degradation and reduce waste products.

In a further embodiment, the analytical system of the invention is a compact MS system that is coupled fluidically with a PCC plant and forms part of its control system. A fluid sample is extracted from the rich or lean solvent streams of the PCC process, or from a point along the absorption column. A sample solution is made-up as necessary and the fluid sample is separated by gas chromatography. The eluent is ionised by means of a ESI source. An atmospheric pressure interface (API) couples the ESI source to the a mass analyser inside a vacuum chamber. The electrospray ions are transported into a vacuum system by means of a vacuum interface and analysed by the mass analyser. Ion current from the mass analyser is collected and measured by an ion counter. The signal from the ion counter is acquired and processed by a computer and used to display mass spectra on an analytical display. The mass analyser may also be operated in selected ion monitoring (SIM) mode where a handful of certain ions are of interest, each representing a chemical species of interest. Mass spectra are used to ‘name’ the chemical species of the sample. The presence of molecular ions in the spectra may be used to identify chemical compounds of interest by means of their molecular mass. The computer may linked to the control system of the PCC and used to transmit data on chemical composition to the control system. The data link may be on-line, forming part of a closed feedback loop, or off-line so that data is monitored by process technicians in a control room. The system of the invention monitors starter materials, intermediate products and reaction products and may provide feedback to a closed-loop control system. The MS system analyzes the chemical composition of the solvent in real-time, thus generating data for the concentration of each chemical present in the mixture. The MS tool continuously measures the relative concentration of starter materials and reaction products and data provided by MS in-line monitoring system is used to measure the efficiency and yield of the capture reaction at any given moment through adjusting process conditions in order to accelerate reactions, to minimise solvent degradation and reduce waste products.

In a further embodiment, the analytical system of the invention is a compact MS system that is coupled fluidically with a PCC plant and forms part of its control system. A fluid sample is extracted from the rich or lean solvent streams of the PCC process, or from a point along the absorption column. A sample solution is made-up as necessary and the fluid sample is separated by liquid chromatography (LC). The eluent is ionised by means of a atmospheric pressure ionisation source. The atmospheric pressure ionisation source may be a suitable soft ionisation source such as ESI, SESI, APCI or APPI that preserves chemical bonds and limits molecular fragmentation, minimising chemical interference, easing interpretation of mass spectra and thereby improving system selectivity. The vacuum interface is an atmospheric pressure interface (API) that couples the atmospheric pressure ionisation source to the a mass analyser inside a vacuum chamber. The ions are transported into a vacuum system by means of a vacuum interface and analysed by the mass analyser. Ion current from the mass analyser is collected and measured by an ion counter. The signal from the ion counter is acquired and processed by a computer and used to display mass spectra on an analytical display. The mass analyser may also be operated in selected ion monitoring (SIM) mode where a handful of certain ions are of interest, each representing a chemical species of interest. Mass spectra are used to ‘name’ the chemical species of the sample. The presence of molecular ions in the spectra may be used to identify chemical compounds of interest by means of their molecular mass. The computer may linked to the control system of the PCC and used to transmit data on chemical composition to the control system. The data link may be on-line, forming part of a closed feedback loop, or off-line so that data is monitored by process technicians in a control room. The system of the invention monitors starter materials, intermediate products and reaction products and may provide feedback to a closed-loop control system. The MS system analyzes the chemical composition of the solvent in real-time, thus generating data for the concentration of each chemical present in the mixture. The MS tool continuously measures the relative concentration of starter materials and reaction products and data provided by MS in-line monitoring system is used to measure the efficiency and yield of the capture reaction at any given moment through adjusting process conditions in order to accelerate reactions, to minimise solvent degradation and reduce waste products.

In a further embodiment, the analytical system of the invention is a compact MS system that is coupled fluidically with a PCC plant and forms part of its control system. A fluid sample is extracted from the rich or lean solvent streams of the PCC process, or from a point along the absorption column. A sample solution is made-up as necessary and the fluid sample is separated by liquid chromatography (LC). The eluent is ionised by means of a ESI source. The vacuum interface is an atmospheric pressure interface (API) that couples the ESI source to the a mass analyser inside a vacuum chamber. The ions are transported into a vacuum system by means of a vacuum interface and analysed by the mass analyser. Ion current from the mass analyser is collected and measured by an ion counter. The signal from the ion counter is acquired and processed by a computer and used to display mass spectra on an analytical display. The mass analyser may also be operated in selected ion monitoring (SIM) mode where a handful of certain ions are of interest, each representing a chemical species of interest. Mass spectra are used to ‘name’ the chemical species of the sample. The presence of molecular ions in the spectra may be used to identify chemical compounds of interest by means of their molecular mass. The computer may linked to the control system of the PCC and used to transmit data on chemical composition to the control system. The data link may be on-line, forming part of a closed feedback loop, or off-line so that data is monitored by process technicians in a control room. The system of the invention monitors starter materials, intermediate products and reaction products and may provide feedback to a closed-loop control system. The MS system analyzes the chemical composition of the solvent in real-time, thus generating data for the concentration of each chemical present in the mixture. The MS tool continuously measures the relative concentration of starter materials and reaction products and data provided by MS in-line monitoring system is used to measure the efficiency and yield of the capture reaction at any given moment through adjusting process conditions in order to accelerate reactions, to minimise solvent degradation and reduce waste products.

In a further embodiment, the analytical system of the invention is a compact MS system that is coupled fluidically with a PCC plant and forms part of its control system. A fluid sample is extracted from the rich or lean solvent streams of the PCC process, or from a point along the absorption column. A sample solution is made-up as necessary and the fluid sample is separated by supercritical fluid chromatography (SFC). The eluent is ionised by means of a atmospheric pressure ionisation source. The atmospheric pressure ionisation source may be a suitable soft ionisation source such as ESI, SESI, APCI or APPI that preserves chemical bonds and limits molecular fragmentation, minimising chemical interference, easing interpretation of mass spectra and thereby improving system selectivity. The vacuum interface is an atmospheric pressure interface (API) that couples the atmospheric pressure ionisation source to the a mass analyser inside a vacuum chamber. The ions are transported into a vacuum system by means of a vacuum interface and analysed by the mass analyser. Ion current from the mass analyser is collected and measured by an ion counter. The signal from the ion counter is acquired and processed by a computer and used to display mass spectra on an analytical display. The mass analyser may also be operated in selected ion monitoring (SIM) mode where a handful of certain ions are of interest, each representing a chemical species of interest. Mass spectra are used to ‘name’ the chemical species of the sample. The presence of molecular ions in the spectra may be used to identify chemical compounds of interest by means of their molecular mass. The computer may linked to the control system of the PCC and used to transmit data on chemical composition to the control system. The data link may be on-line, forming part of a closed feedback loop, or off-line so that data is monitored by process technicians in a control room. The system of the invention monitors starter materials, intermediate products and reaction products and may provide feedback to a closed-loop control system. The MS system analyzes the chemical composition of the solvent in real-time, thus generating data for the concentration of each chemical present in the mixture. The MS tool continuously measures the relative concentration of starter materials and reaction products and data provided by MS in-line monitoring system is used to measure the efficiency and yield of the capture reaction at any given moment through adjusting process conditions in order to accelerate reactions, to minimise solvent degradation and reduce waste products.

These and other features and benefit will be understood with reference to the following exemplary embodiments.

### BRIEF DESCRIPTION OF THE DRAWINGS

FIG. 1 is a schematic of a typical solvent-based PCC process as known in prior art FIG. 2 is a schematic of part of the absorption column of solvent-based PCC process

FIG. 3 is diagram of the system of the invention describing a MS coupled to a sample solution by soft ionisation source

FIG. 4 is diagram of the system of the invention describing a MS coupled to a sample solution by a chromatography module and a soft ionisation source

FIG. 5 is a diagram of an embodiment of the online analytical system of the invention with a sample injector means, a sample loop and a chromatography module

FIG. 6 is a schematic of an online analytical system which forms part of the control system of the PCC process, and includes a soft ionisation source, and a vacuum interface.

FIG. 7 is a schematic of an online analytical system which forms part of the control system of the PCC process, and includes a soft ionisation source, an IMS separator and a vacuum interface.

FIG. 8 is a schematic of an online analytical system which forms part of the control system of the PCC process, and includes a GC, a atmospheric pressure ionisation source and a API.

FIG. 9 is a schematic of an embodiment of an online analytical system which forms part of the control system of the PCC process, and includes a GC, an ESI source and a API.

FIG. 10 is a schematic of an online analytical system which forms part of the control system of the PCC process, and includes a LC, an atmospheric pressure ionisation source and a API.

FIG. 11 is a schematic of an online analytical system which forms part of the control system of the PCC process, and includes a LC, an ESI source and an API.

FIG. 12 is a schematic of an online analytical system which forms part of the control system of the PCC process, and includes a SFC, an atmospheric pressure source and an API.

### DETAILED DESCRIPTION OF THE INVENTION

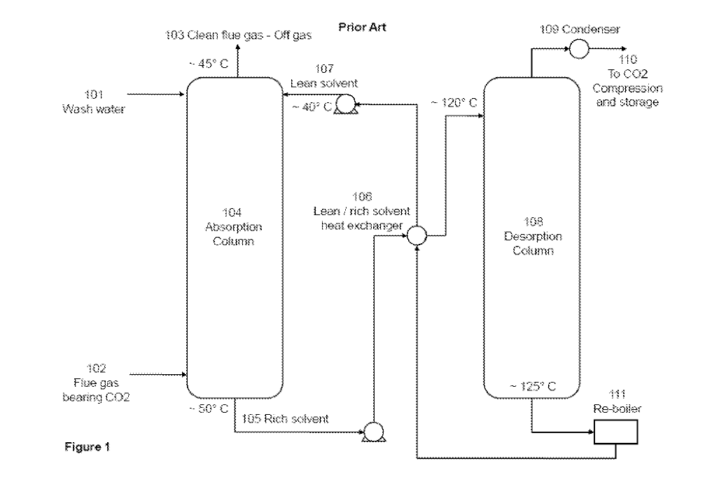

A detailed description of preferred exemplary embodiments in accordance with the present teaching is provided with reference to FIGS. 1 to 12. It will be understood that these are provided to assist the person of skill in the art with an understanding of the present teaching and it is not intended to limit the scope to that hereinafter described.

Shown in FIG. 1 is a typical post-combustion capture (PCC) process of the prior art. Flue gases 102 bearing CO₂ are introduced into the bottom of an absorption column 104 where they are mixed with lean solvent 107 and water 101. The solvent-based mixture is chemically loaded with the CO₂ of the flue gas as it passes through the column 104 until it leaves as a CO₂ rich solvent 105 from the bottom. The rich solvent stream 105 is pumped through a heat exchanger 106 where it is heated by a hot, lean stream from the re-boiler 111. The rich solvent enters a desorption column 108 where it gives up CO₂ to a condenser 109 for compression, storage and transportation 110.

In FIG. 2 an absorption column 204 of the PCC of the prior art is shown. Samples may be taken from various points in the process as part of a scheme to monitor solvent quality. At a minimum, samples are taken from the lean solvent stream 207 into the column 204 and from the rich, or loaded, solvent stream 205 out of the column 204. To date these samples are collected infrequently and analysed off-line in remote analytical laboratories. Using a system and methodology in accordance with the present teaching it is possible to interface directly with the PCC process so as to allow samples to be taken frequently, or continuously, from the PCC process using online analytical systems 208 and 209 from one or both of the lean stream 207 and rich stream 205. By providing a suitably compact monitoring system samples may be taken along the absorption column by multiple monitoring systems (e.g. 208, 209 and 201 to 214), or by multiplexing one of more online monitoring instruments to multiple sample points.

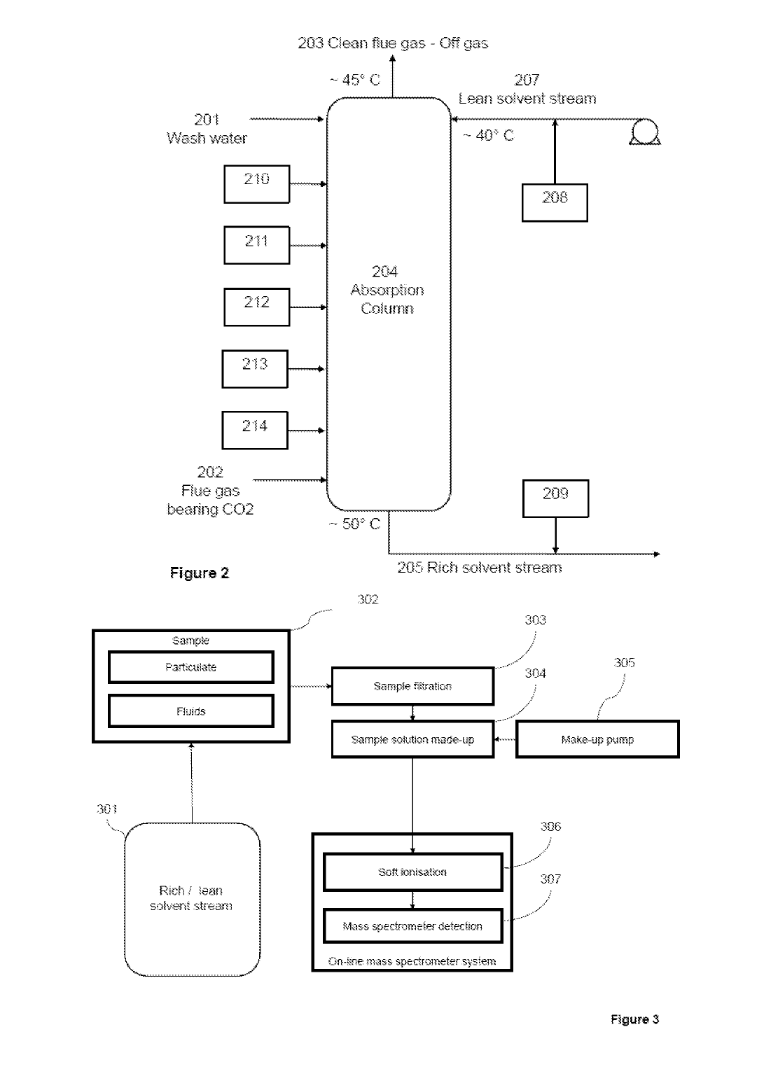

FIG. 3 shows in schematic form a monitoring system in accordance with the present teaching. A fluid sample 302 is extracted from a solvent stream 301. The sample 302 may comprise particulate suspended in a fluid mixture and may require filtration using an inline filter 303. The filter may be a pre-column, granular packing or mesh. The sample may require dilution prior to analysis so a solution may be made up 304 by means of a solvent reservoir and make-up pump 305. The make-up pump may be a simple infusion pump infusing a solvent from a syringe into a sample stream via a mixer or suitable six-port valve and sample loop. The sample solution is introduced to a soft ionisation source 306. These ions are directed to a mass spectrometer (MS) 307 for identification by means of their mass to charge ratios. A more detailed schematic of the MS is shown in FIG. 5.

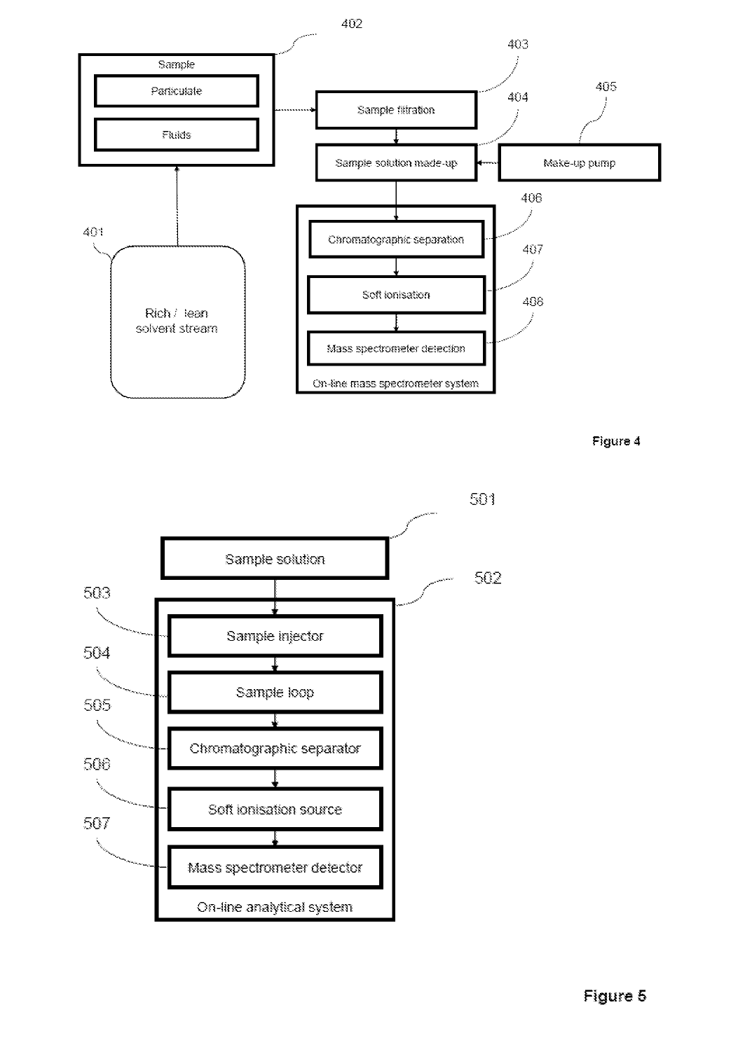

Another exemplary arrangement is described in FIG. 4. A fluid sample 402 is extracted from a solvent stream 401 which may be rich or lean, or from the absorption column 204. The sample 402 may comprise particulate suspended in a fluid mixture and therefore may require filtration using an inline filter 403. The filter may be a pre-column, granular packing or mesh. The sample may require dilution prior to analysis so a solution may be made up 404 by means of a solvent reservoir and make-up pump 405. The sample solution is introduced to a chromatographic separation module 406. A soft ionisation source 407 couples the chromatography module 406 to the mass spectrometer 408. Ions are generated as species elute from the chromatography module 406 by the soft ionisation source 407. The ions directed to a mass spectrometer 408 for identification by means of their mass to charge ratios. A more detailed schematic of the MS is shown in FIG. 5.

A more detailed schematic of a system provided in accordance with the present teaching is shown in FIG. 5. A sample solution 501 is made up as described in FIG. 3 and introduced to an online analytical system 502 via the interface of a sample injector 503. The sample injector 503 may be a simple syringe pump. A sample loop 504 collects a known volume of sample solution 501 prior to injection onto chromatographic separator 505. The sample loop 504 may form part of a six-port valve. The chromatographic separator 505 may be suitable chromatography column. The mixture of the sample solution 501 is separated and purified by the separator 505 and eluted into a soft ionisation source 506 where the species are individually ionised. A mass spectrometer detector 507 receives ions and analysing them by their mass to charge ratios before collecting ion current, acquiring, amplifying and processing this signal and displaying the results as a mass spectrum. The spectrum may be used to identify the chemical species by the mass to charge ratios of the molecular ions and their fragmentation patterns.

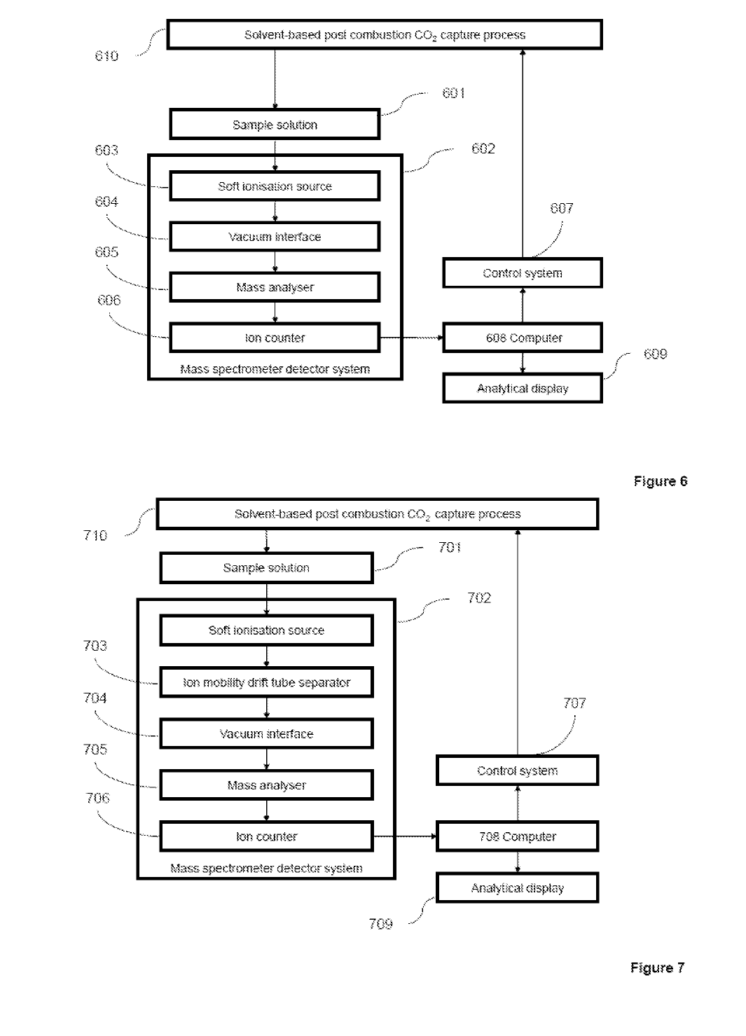

In another embodiment the system forms part of the control system of the PCC and such an exemplary arrangement is shown in FIG. 6. A sample is extracted and made-up into a suitable solution 601 as described in for example FIG. 3. Soft ionisation 603 generates a beam of ions for transport through a vacuum interface 604 for coupling to a mass analyser 605. The mass analyser 605 may be an ion trap, time of flight, quadrupole, triple quadrupole, magnetic sector, orbital ion trap, linear ion trap, rectilinear ion trap, cross-field, cycloidal or rotational field mass analyser. Ions are filtered by mass to charge ratio in the analyser 605 and ion current is collected by an ion counter 606. The ion counter 606 may be channeltron, electron multiplier, dynode converter, photomultiplier tube, avalanche photo diode, microchannel plate, faraday plate or some suitable collector. Ion counts are converted to signal and processes and displayed by a computer 608 on an analytical display 609 such as total ion chromatograms, mass spectra, selected ion chromatograms, extracted ion chromatograms etc. The mass spectrometer detector 602 data may be relayed to a control system 607 where is used as feedback to typical process 610 conditions such as temperature, flow, pH, solvent dilution, solvent replenishment etc. The data may be exploited online in real-time as part as of a closed loop feedback control system, or off line by operators in the control room of the PCC.

In FIG. 7 another embodiment is described where the analytical system is a compact MS system 702 that is coupled fluidically with a PCC plant 710 and forms an input to its control system 707, but which utilises separation by ion mobility 703. A fluid sample 701 is extracted from the rich or lean solvent streams of the PCC process 710, or from a point along the absorption column. A sample solution is made-up as necessary 701 and the fluid sample is ionised by means of a soft ionisation source 702. The soft ionisation source 702 preserves chemical bonds and limits molecular fragmentation, minimising chemical interference, easing interpretation of mass spectra and thereby improving system selectivity. Ions generated by the soft ionisation source 702 may be separated by their drift time along the drift tube of an ion mobility spectrometer (IMS) 703. The IMS 703 effects some separation of the ions by means of permitting them to drift in a strong, a potentially varying, electric field. The IMS 703 may be a field-asymmetric ion mobility spectrometer (FAIMS). A vacuum interface 704 couples the IMS to the a mass analyser 705 inside a vacuum chamber. The ions are transported into a vacuum system by means of a vacuum interface 704 and analysed by the mass analyser 705. The vacuum interface 704 may be differentially pumped. Ion current from the mass analyser 705 is collected and measured by an ion counter 706. The signal from the ion counter 706 is acquired and processed by a computer 708 and used to display mass spectra on an analytical display 709. The mass analyser 705 may also be operated in selected ion monitoring (SIM) mode where a handful of ions are of interest, each representing a certain species of interest. Mass spectra are used to ‘name’ the chemical species of the sample. The presence of molecular ions in the spectra may be used to identify chemical compounds of interest by means of their molecular mass. The computer 708 may linked to the control system 707 of the PCC 710 and used to transmit data on chemical composition to the control system. The data link may be on-line, forming part of a closed feedback loop, or off-line so that data is monitored by process technicians in a control room. The system of the invention may be used to monitor starter materials such as amine and water, intermediate products and reaction products such as carbamates and may provide feedback to a closed-loop control system 707. The MS system 702 analyzes the chemical composition of the solvent sample 701 in real-time, thus generating data for the concentration of each chemical present in the mixture.

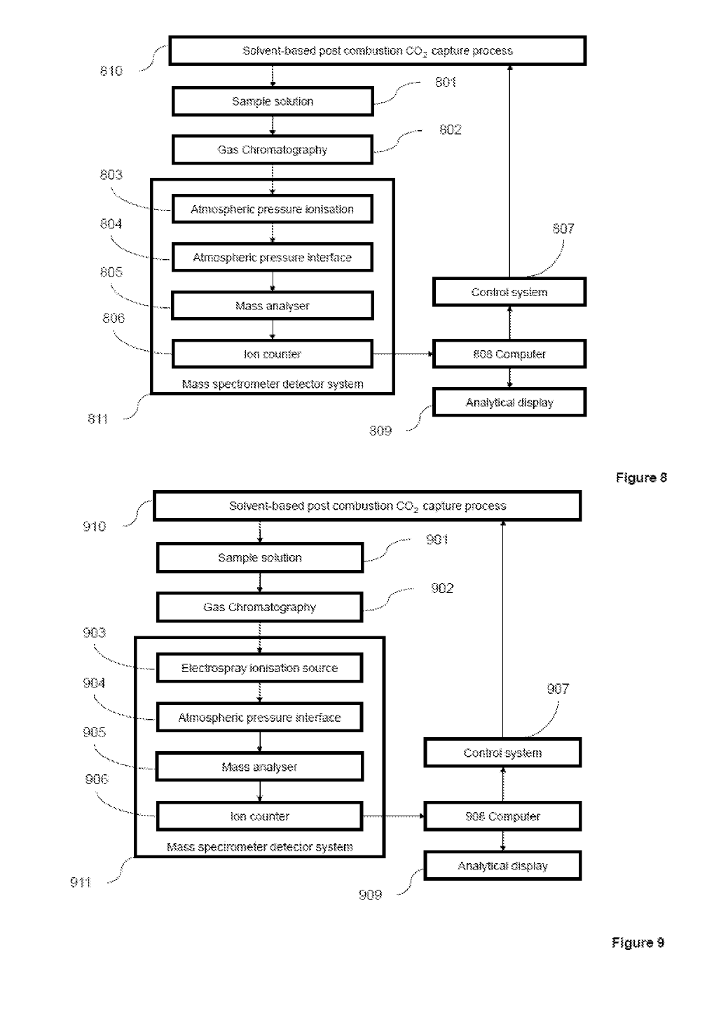

In FIG. 8 the analytical system is shown in an exemplary arrangement as being a compact MS system 811 that is coupled fluidically with a PCC plant 810 and forms part of its control system 807, but which also makes use of GC separation 802. A fluid sample is extracted from the rich or lean solvent streams of the PCC process 810, or from a point along the absorption column. A sample solution 801 is made-up as necessary and the fluid sample is separated by gas chromatography 802. The eluent is ionised by means of a atmospheric pressure ionisation source 803. The atmospheric pressure ionisation source 803 may be a suitable soft ionisation source such as ESI, SESI, APCI or APPI that preserves chemical bonds and limits molecular fragmentation, minimising chemical interference, easing interpretation of mass spectra and thereby improving system selectivity. The vacuum interface 804 is an atmospheric pressure interface (API) that couples the atmospheric pressure ionisation source 803 to the a mass analyser inside a vacuum chamber. The API 804 is preferably differentially pumped. The ions are transported into a vacuum system by means of the API 804 and analysed by the mass analyser 805. Ion current from the mass analyser 805 is collected and measured by an ion counter 806. The signal from the ion counter 806 is acquired and processed by a computer 808 and used to display mass spectra on an analytical display 809. The mass analyser 805 may also be operated in selected ion monitoring (SIM) mode where a handful of certain ions are of interest, each representing a chemical species of interest. Mass spectra are used to ‘name’ the chemical species of the sample. The presence of molecular ions in the spectra may be used to identify chemical compounds of interest by means of their molecular mass. The MS system 811 computer 808 may linked to the control system 807 of the PCC 810 and used to transmit data on chemical composition to the control system 807. The data link may be on-line, forming part of a closed feedback loop, or off-line so that data is monitored by process technicians in a control room and used in decision making.

In FIG. 9 another embodiment of the analytical system is shown. A compact MS system 911 that is coupled fluidically with a PCC plant 910 and forms part of its control system 907 but which utilises GC separation 902 and a ESI source 903. A sample solution 901 is made-up as necessary and the fluid sample is separated by gas chromatography 902. The eluent is ionised by means of a ESI source 903. An API 904 couples the ESI source 903 to the a mass analyser 905 inside a vacuum chamber. Ion current from the mass analyser is collected and measured by an ion counter 906. The signal from the ion counter is acquired and processed by a computer 908 and used to display mass spectra on an analytical display 909. The mass analyser may also be operated in selected ion monitoring (SIM) mode as before. Mass spectra are used to ‘name’ the chemical species of the sample. The computer 908 may linked to the control system 907 of the PCC 910 and used to transmit data on chemical composition to the control system. The data link may be on-line, forming part of a closed feedback loop, or off-line so that data is monitored by process technicians in a control room.

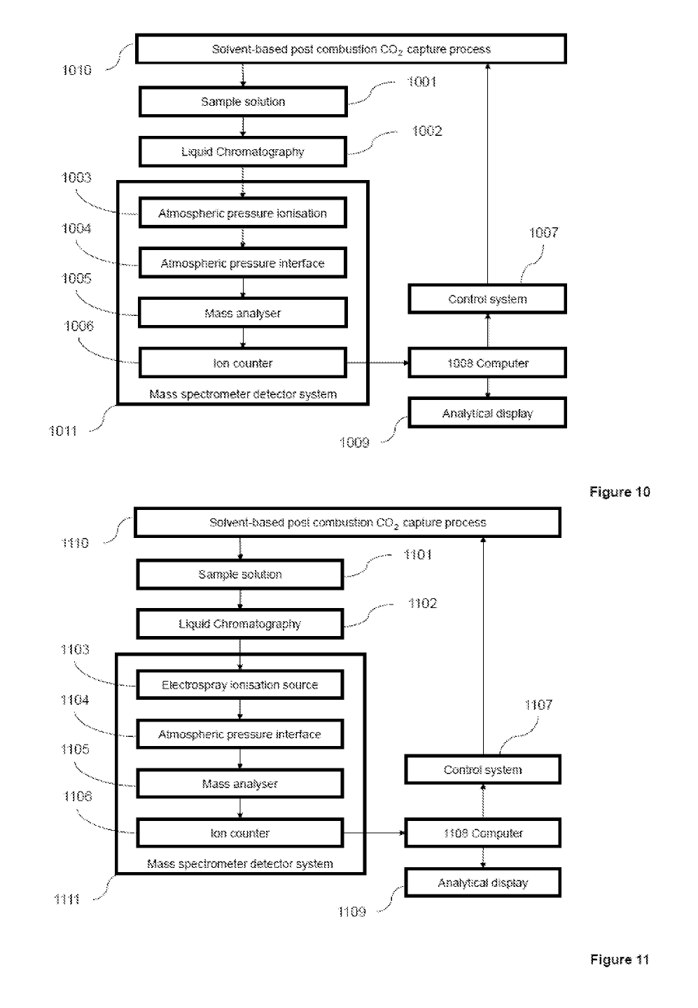

Another embodiment is featured in FIG. 10. The analytical system of this arrangement is a compact MS system 1011 that is coupled fluidically with a PCC plant 1010 and forms part of its control system 1007. A sample solution 1001 is made-up as necessary and the fluid sample is separated by liquid chromatography (LC) 1002. The eluent is ionised by means of a suitable atmospheric pressure ionisation source 1003. The atmospheric pressure ionisation source 1003 may be a suitable soft ionisation source such as ESI, SESI, APCI or APPI that preserves chemical bonds and limits molecular fragmentation, minimising chemical interference, easing interpretation of mass spectra and thereby improving system selectivity. The atmospheric pressure interface (API) 1004 couples the atmospheric pressure ionisation source to the a mass analyser 1005 inside a vacuum chamber. Ion current from the mass analyser is collected and measured by an ion counter 1006. The signal from the ion counter is acquired and processed by a computer 1008 and used to display mass spectra on an analytical display 1009. The computer 1008 of the system 1011 may linked to the control system of the PCC 1010 and used to transmit data on chemical composition to the control system.

In FIG. 11 a further preferred embodiment is depicted wherein the analytical system is a compact MS system 1111 that is coupled fluidically with a PCC plant 1110 and forms part of its control system 1107, but wherein a sample solution is made-up 1101 and the fluid sample is separated by liquid chromatography (LC) 1102 and ionised by means of a ESI source 1103. The API 1104 couples the ESI source 1103 to the a mass analyser 1105 inside a vacuum chamber. Ion current from the mass analyser is collected and measured by an ion counter 1106. The signal from the ion counter is acquired and processed by a computer 1108 and used to display mass spectra on an analytical display 1109. The computer 1108 may linked to the control system of the PCC 1110 and used to transmit data on chemical composition to the control system 1110.

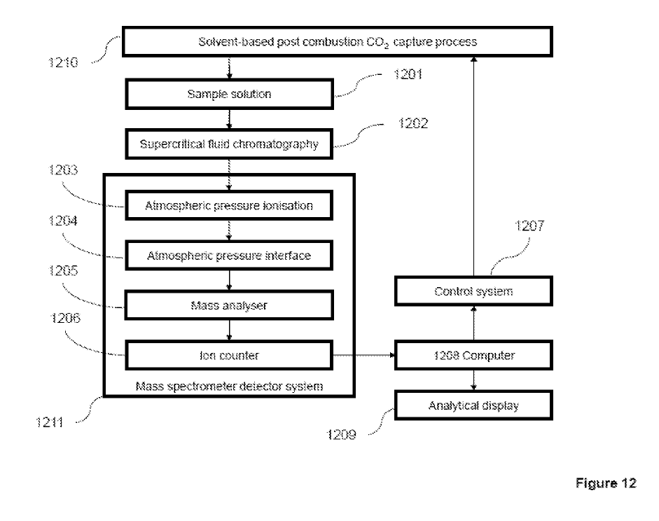

In FIG. 12 another preferred embodiment is shown wherein the analytical system is a compact MS system 1211 is coupled fluidically with a PCC plant 1210 and forms part of its control system 1207 but wherein a sample solution 1201 is made-up and is separated by supercritical fluid chromatography (SFC) 1202. The eluent is ionised by means of a atmospheric pressure ionisation source 1203 such as a suitable soft ionisation source such as ESI, SESI, APCI or APPI that preserves chemical bonds and limits molecular fragmentation, minimising chemical interference, easing interpretation of mass spectra and thereby improving system selectivity. The vacuum interface is an atmospheric pressure interface (API) 1204 that couples the atmospheric pressure ionisation source 1203 to the a mass analyser 1205 inside a vacuum chamber. Ion current from the mass analyser is collected and measured by an ion counter 1206. The signal from the ion counter is acquired and processed by a computer 1208 and used to display mass spectra on an analytical display 1209. The computer 1208 may linked to the control system of the PCC and used to transmit data on chemical composition to the control system 1207.

It will be appreciated and understood that what has been described herein are exemplary arrangements of an analysis tool that is directed towards real-time analysis of carbon capture processes which may be generally considered as including any fluid that chemically binds with greenhouse gases in flue streams such as methane and CO₂. By employing a soft ionisation source such as the exemplary atmospheric ionisation sources that effect ionisation of the sample in non-vacuum conditions, the chromatographic flow rate is not limited by the pumping speed of the vacuum pumps and the column may have a higher flow rate permitting more rapid separation and a shorter system response time. Soft ionisation, i.e. the formation of ions without breaking chemical bonds, is particularly advantageous in the context of the chemically complex samples as described herein in that soft ionisation advantageously produces one ‘molecular ion’, whose mass to charge ratio or time of flight corresponds to its molecular weight, and has is a faster and easier means of identifying eluted compounds. The separation of the fluid into its chemical constituents has been described with reference to the exemplary use of a chromatography column that could be a gas, liquid or supercritical fluid based chromatography module. However it is possible to separate mixtures using other separation techniques such as ion mobility or capillary electrophoresis and the use of such techniques should be considered within the context of the separation module described herein.

It will be appreciated that samples from PCC processes may be ‘messy’. Due to the complex chemical matrix that is a carbon-capture solvent, lengthy chromatographic separation times are required to ensure adequate separation and purification of all the compounds in the mixture. Gas chromatographic (GC) retention times of several minutes may be required before all the components of have eluted from the GC column. In fact, samples of interest may contain hundreds of components. While users may not need to separate and identify all of the components during operation, nonetheless an analytical solution will need to rapidly separate and analyse complex samples and identify their components. In the context of capture operations, when processing hundreds of tonnes of flue gasses, the cost of delays and missed opportunities would be very high. To address these problems there is provided in accordance with the present teaching, an analytical tool and methodology that would provide rapid response times. To achieve this improved response rate, the tool advantageously employs a chromatographic solution featuring a faster flow rate and shorter separation times than heretofore possible in process solvent analysis. By providing for ionisation of the sample in non-vacuum conditions, i.e. at atmospheric pressure, then the gas chromatographic (GC) flow rate is not limited by the pumping speed of the vacuum pumps and the GC column may have a higher flow rate permitting more rapid separation and a shorter system response time.

It will be appreciated that traditionally where a chromatographic column is used to separate a mixture, a mass spectrometer (MS) detector is used to identify the compounds as they elute. The MS detector is a vacuum instrument and generally features an ion source inside the vacuum chamber to which the GC column is coupled and which ionises molecules of each constituent compound as they elute from the column. Typical ion sources used with GC are electron ionisation (EI) and chemical ionisation (CI). Both EI and CI take place inside the vacuum chamber and involve bombarding eluted molecules with energetic electrons or ions, fragmenting the neutral molecules and producing charged particles (i.e. ions). This fragmentation adds further complexity where some many chemicals are concerned, leading to mass spectral interpretation and further delays. Problems arise when component co-elute from the column and fragments over-lap. Over-lapping fragments can make it impossible to separate mass spectra and identify compounds. Co-eluting compounds will be a problem when separations are accelerated by increasing flow rate or temperature ramp for example. To address these shortcomings of previous systems, a system in accordance with the present teaching employs a ‘soft’ ionisation source that does not fragment chemical species but which instead produces one ‘molecular ion’, whose mass to charge ratio corresponds to it molecular weight, is a faster and easier means of identifying eluted compounds. The use of soft ionisation permits identification of compounds during rapid separation of compounds. Such a ‘soft’ ionisation processes may be conducted outside the GC vacuum chamber at elevated pressures and include those provided by techniques such as atmospheric pressure glow discharge ionisation (APGDI), atmospheric pressure corona discharge ionisation (APCDI), atmospheric pressure chemical ionisation (APCI), electrospray ionisation (ESI), atmospheric pressure photo ionisation (APPI), desorption electrospray ionisation (DESI), secondary electrospray ionisation (SESI) and so on.

While the specifics of the mass spectrometer have not been described herein a miniature instrument such as that described herein may be advantageously manufactured using microengineered instruments such as those described in one or more of the following co-assigned US applications: U.S. patent application Ser. No. 12/380,002, U.S. patent application Ser. No. 12/220,321, U.S. patent application Ser. No. 12/284,778, U.S. patent application Ser. No. 12/001,796, U.S. patent application Ser. No. 11/810,052, U.S. patent application Ser. No. 11/711,142 the contents of which are incorporated herein by way of reference. Within the context of the present invention the term microengineered or microengineering or micro-fabricated or microfabrication is intended to define the fabrication of three dimensional structures and devices with dimensions in the order of millimetres or sub-millimetre scale.

Where done at micron-scale, it combines the technologies of microelectronics and micromachining. Microelectronics allows the fabrication of integrated circuits from silicon wafers whereas micromachining is the production of three-dimensional structures, primarily from silicon wafers. This may be achieved by removal of material from the wafer or addition of material on or in the wafer. The attractions of microengineering may be summarised as batch fabrication of devices leading to reduced production costs, miniaturisation resulting in materials savings, miniaturisation resulting in faster response times and reduced device invasiveness. Wide varieties of techniques exist for the microengineering of wafers, and will be well known to the person skilled in the art. The techniques may be divided into those related to the removal of material and those pertaining to the deposition or addition of material to the wafer. Examples of the former include:

Wet chemical etching (anisotropic and isotropic)

Electrochemical or photo assisted electrochemical etching

Dry plasma or reactive ion etching

Ion beam milling

Laser machining
Excimer laser machining

### Electrical Discharge Machining

Whereas examples of the latter include:

Evaporation

Thick film deposition

Sputtering

Electroplating

Electroforming

Moulding

Chemical vapour deposition (CVD)

Epitaxy

While exemplary arrangements have been described herein to assist in an understanding of the present teaching it will be understood that modifications can be made without departing from the spirit and or scope of the present teaching. To that end it will be understood that the present teaching should be construed as limited only insofar as is deemed necessary in the light of the claims that follow.

Furthermore, the words comprises/comprising when used in this specification are to specify the presence of stated features, integers, steps or components but does not preclude the presence or addition of one or more other features, integers, steps, components or groups thereof.

What is claimed is:

1. An in-line analysis system for direct analysis of solvents used to chemically bind with CO₂ present in flue gases within a post-combustion CO₂ capture system, the analysis system comprising:
a fluid interface for extracting a sample from a fluid stream in the post combustion CO₂ capture system;
a mass spectrometer coupled to the interface, the mass spectrometer configured to selectively identify chemical components of the fluid stream by detection of their molecular ions.

2. The analysis system of claim 1 wherein the mass spectrometer comprises
a. an atmospheric pressure ionisation source coupled to
b. a mass analyser,
wherein the mass analyser identifies the chemical components of the fluid stream by their molecular ions as they are ionised by the atmospheric pressure ionisation source.

3. The analysis system of claim 2 wherein the ionisation source is a soft ionisation source configured to effect the formation of ions without breaking chemical bonds.

4. The analysis system of claim 3 comprising a chromatographic separation module provided between the fluid interface and the soft ionisation source, the soft ionisation source coupling the chromatographic module to the mass analyser such that operably ions are generated as species elute from the chromatographic module by the soft ionisation source prior to introduction into the mass analyser.

5. The analysis system of claim 3 comprising an ion mobility separation module provided between the soft ionisation source and the mass analyser, the ion mobility separation module operably effecting a separation of ions based on their drift time prior to introduction into the mass analyser.

6. The analysis system of claim 1 comprising a filter provided between the fluid interface and the mass spectrometer.

7. The analysis system of claim 1 comprising a dilutor provided downstream of the fluid interface to selectively effect a dilution of sample received from the fluid stream prior to analysis.

8. The analysis system of claim 4 comprising a sample loop provided prior to the chromatographic module.

9. The analysis system of claim 8 wherein the sample loop is configured for operably providing a pre-concentration of a species of interest prior to discharge to the chromatographic module.

10. The analysis system of claim 8 wherein the sample loop comprises a sorbent trap.

11. The analysis system of claim 2 comprising a vacuum interface disposed between the ionisation source and the mass analyser.

12. The analysis system of claim 2 comprising an atmospheric interface disposed between the ionisation source and the mass analyser.

13. The analysis system of claim 2 wherein the mass analyser is coupled to an ion counter such that ions are filtered by their mass to charge ratios in the mass analyser and impact the ion counter generating an electrical current.

14. The analysis system of claim 2 wherein the ionisation source is an electrospray ionisation source.

15. The analysis system of claim 4 wherein the chromatographic module comprises a gas chromatography column.

16. The analysis system of claim 4 wherein the chromatographic module comprises a liquid chromatographic column or a supercritical fluid chromatographic column.

17. The analysis system of claim 2 wherein the mass analyser is a microengineered based analyser.

18. The analysis system of claim 1 provided in a control loop configuration within the post-combustion CO₂ capture system.

19. The analysis system of claim 1 wherein the fluid interface provides for extraction of the fluid sample from one of a rich or lean solvent stream of the post-combustion CO₂ capture system.

20. The analysis system of claim 1 wherein the fluid interface provides for extraction of the fluid sample from one or more points along an absorption column provided within the post-combustion CO₂ capture system.

21. The analysis system of claim 1 configured to monitor for changes in the composition of solvents such as monoethanolamine (MEA), AEPD (2-amino-2-ethyl-1,3-propanediol), AMP (2-amino-2-methyl-1-propanol), AMPD (2-amino-2-methyl-1,3-propanediol), DEA (diethanolamine), MDEA (methyldiethanolamine), PZ (piperazine) and THAM (tris-(hydroxymethyl)aminomethane) within the post-combustion CO₂ capture system.

22. The analysis system of claim 1 comprising a sample loop.

23. A method of directly analysing solvents used to chemically bind with CO₂ present in flue gases within a post-combustion CO₂ capture system, the method comprising:
Using a fluid interface to extract a sample from a fluid stream in the post-combustion CO₂ capture system;
introducing the extracted sample into a mass spectrometer that is coupled to the fluid interface, the mass spectrometer being configured to selectively identify chemical components of the fluid stream by detection of their molecular ions.

24. An in-line method of directly analysing solvents used within a post-combustion CO₂ capture system, the solvents being used to chemically bind with CO₂ present in flue gases within the post-combustion CO₂ capture system, the method comprising using the system of claim 1 in effecting an analysis of the constituents of the solvents so as to determine their efficacy in CO₂ extraction.

25. The method of claim 24 wherein samples are extracted from the fluid stream and passed directly to the mass spectrometer, the mass spectrometer being in fluid communication with the fluid interface.

26. The method of claim 24 further comprising using the analsysis of the constituents of the solvents in a closed-loop control of the CO₂-capture process.

27. The method of claim 26 comprising using real-time compositional data of the solvent constituents in a feedback loop to adjust parameters such as temperature, flow, pH, solvent dilution, solvent replenishment, flow rates and pressure within the CO₂ capture process to optimise the efficiency of the system.
